(19) United States
(12) Patent Application Publication
(10) Pub. No.: US 2004/0193173 A1
(43) Pub. Date: Sep. 30, 2004

(54) Sleeve handle and sleeve system

(76) Inventors: Christian Knopfle, Donaueschingen (DE); Reinhard Rubecamp, Malterdingen (DE); Gregg Diamond, Charlotte, NC (US)

Correspondence Address: HOWARD & HOWARD ATTORNEYS, P.C., THE PINEHURST OFFICE CENTER, SUITE #101, 39400 WOODWARD AVENUE, BLOOMFIELD HILLS, MI, 48304-5151, US

(21) Appl. No.: 10/758,907

(22) Filed: Jan. 16, 2004

(30) Foreign Application Priority Data
Jan. 17, 2003 (DE) 103 01 691.0

(51) Int. Cl. A61B 17/58

(52) U.S. Cl. 606/96

(57) ABSTRACT

The invention relates to a sleeve system for use in combination with a plurality of different types of sleeve for surgical operations, particularly in the central region of the face. Said sleeve system comprises a sleeve handle, which is constructed for the purpose of mounting different types of sleeve, and a first arrangement for interacting with a mounted sleeve of a first type, and also a second arrangement for interacting with a mounted sleeve of a second type. The first arrangement prevents rotation of a sleeve of the first type, relative to the handle, and the second arrangement makes possible guided rotation of a mounted sleeve of the second type, relative to said handle.

### TECHNICAL FIELD

The invention relates to a sleeve handle for use in combination with a plurality of different types of sleeve for surgical operations, particularly in the central region of the face. The invention also relates to a sleeve system which comprises the handle and the plurality of different types of sleeve.

### BACKGROUND TO THE INVENTION

A sleeve system for surgical operations is known from DE 197 17 977 A1. It comprises a handle and also a tubular sleeve which can be fastened to an outer end of said handle. In the context of a surgical operation, the sleeve can be used for various purposes. Thus, for example, said sleeve may serve as a drill-guiding device in a preliminary drilling operation for a fastening screw. The fastening screw can then be reliably positioned by means of the sleeve. Said sleeve can also function as a positioning aid for a screwdriver blade in the course of subsequent screwing-in of the bone screw.

In order to be able to perform the aforesaid steps in a precise manner, a secure connection between the sleeve handle and the mounted sleeve is necessary. In DE 197 17 977 A1, a gripping piece is provided for this purpose. The sleeve is fastened to the sleeve handle by means of said gripping piece.

The underlying object of the -invention is to indicate a multifunctional sleeve handle and also a multifunctional sleeve system.

### SUMMARY OF THE INVENTION

In order to achieve this object, the invention proposes a sleeve handle for use in combination with a plurality of different types of sleeve. A mounting end of the handle is constructed for mounting the different types of sleeve and is constructed with a first arrangement for interacting with a mounted sleeve of a first type, and also a second arrangement for interacting with a mounted sleeve of at least one second type. The first arrangement prevents rotation of a sleeve of the first type, relative to the handle, and the second arrangement permits guided rotation of a mounted sleeve of a second type, relative to said handle.

In addition to the handle, the sleeve system according to the invention comprises a plurality of different types of sleeve. The handle or a sleeve of a first type is provided with a first arrangement which prevents rotation of a mounted sleeve of the first type, relative to the handle. In addition, the handle or a sleeve of a second type possesses a second arrangement which makes possible guided rotation of a mounted sleeve of the second type, relative to the handle.

A sleeve handle that can be used in combination with a plurality of different types of sleeve permits multifunctional use. This multifunctionality is aided by the fact that it is possible to couple sleeves to a specific end of the handle in either a rotatable or a rotation-proof manner, according to their type.

Thus, it is possible to couple to the handle, in a rotation-proof manner, sleeves of a first type each having a through-aperture which is concentric with respect to an imaginary axis of rotation. On the other hand, sleeves of a second type each having a through-aperture which is eccentric with respect to an axis of rotation may be constructed so as to be capable of being coupled to the handle in a rotatable manner. The imaginary axis of rotation of the sleeves of the first type refers to that axis about which the sleeves of the first type would be rotatable if the first arrangement already mentioned, which prevents rotation of a mounted sleeve relative to the handle, were not present.

The first arrangement may be constructed so as to interact with a complementary arrangement in a form-locking or friction-locking manner for the purpose of preventing a sleeve from being capable of rotating. It is possible to construct the first arrangement as a blocking element in the form of, for example, at least one recess or at least one projection, which blocking element interacts, for the purpose of eliminating the rotation of a mounted sleeve of the first type, with a complementary blocking element on said sleeve. If the first arrangement possesses, for example, a blocking element in the form of a recess, the complementary blocking element is advantageously constructed as a projection that engages in the said recess, and vice versa.

According to one configuration of the invention, the first arrangement makes possible rotation-proof fixing of a sleeve of the first type in various angular positions, with respect to the sleeve handle, which angular positions may be any desired ones or, expediently, predetermined ones. For this purpose, the first arrangement may possess a plurality of blocking elements which are spaced apart in the peripheral direction of an imaginary axis of rotation. In this case, a specific angular position of a sleeve with respect to the sleeve handle can be realised through the fact that the complementary blocking element on the sleeve interacts with one specific blocking element of the plurality of blocking elements that are spaced apart in the peripheral direction of the imaginary axis of rotation.

The second arrangement already mentioned, which permits guided rotation of a mounted sleeve of the second type, relative to the handle, may be of different construction. The second arrangement preferably comprises a guide element in the form of, for example, at least one guide projection or at least one guide groove, which guide element is constructed so as to interact with a complementary guide element on a sleeve of the second type, for example in a form-locking manner. If the second arrangement possesses, for instance, a guide element constructed as a guide groove, that guide element on the sleeve which is complementary thereto may be constructed as a guide projection that engages in the said guide groove, and vice versa. Said guide projection is expediently pretensioned in the direction of the guide groove.

As already explained, the sleeve handle permits the mounting of different types of sleeve. For this purpose, a third arrangement may be provided at an end of the handle that serves to mount the different types of sleeve. The third device has the function of coupling the sleeves of the first and/or second type to the handle in a captive manner. However, this coupling function is preferably undertaken, at least in the case of the sleeves of the second type, by the second arrangement which has been explained above. In other words, the second arrangement not only functions, in an expedient manner, as a guide with regard to the rotation of a mounted sleeve of the second type, but at the same time guarantees captive mounting of a sleeve of the second type at the mounting end of the handle. According to a preferred variant of the invention, the second arrangement additionally interacts with the sleeves of the first type in such a way that it also guarantees captive mounting of a sleeve of the first type at the mounting end of the handle. In this case, the functionality of the first arrangement may be confined to merely preventing relative movement between the handle and a sleeve of the first type. In principle, however, it would also be possible to develop the first arrangement to the effect that it not only permits rotation-proof coupling of a sleeve of the first type to the handle but, in addition, makes possible captive mounting of a sleeve of the first type at the mounting end of the handle.

According to one development of the invention, the handle possesses, at its mounting end, a through-aperture into which the sleeves of different type can be introduced. When the sleeve handle is configured in such a way, the second arrangement may be disposed in the region of a side wall of the through-aperture. The sleeves expediently possess an enlargement in diameter which functions as a stop and delimits the length over which the sleeves are introduced into the through-aperture in the handle. In this case, a pressing force which is directed into the stop by means of the handle can be safely transmitted to the proximal end of a mounted sleeve, i.e. to the end that faces towards the operating area.

Although the above description of details of the first and second arrangements has referred primarily to the situation in which said arrangements are constructed in the region of the handle, it is also possible, according to the invention, to dispose the above described arrangements on the sleeves.

### BRIEF DESCRIPTION OF THE DRAWINGS

Further advantages and configurations of the invention emerge from the following description of preferred exemplified embodiments and from the drawings, in which:

FIG. 1 shows a perspective view of a sleeve handle according to the invention, with an eccentric sleeve mounted therein;

FIG. 2 shows a perspective view of a mounting end of the sleeve handle represented in FIG. 1, and also of an eccentric sleeve;

FIG. 3 shows an enlarged perspective view of the mounting end of the sleeve handle represented in FIG. 1;

FIG. 4 shows an enlarged perspective view of the eccentric sleeve illustrated in FIG. 2;

FIG. 5 shows a perspective view of the mounting end of the sleeve handle represented in FIG. 1, and of a concentric sleeve;

FIG. 6 shows a side view of the components in FIG. 5; and

FIG. 7 shows an enlarged perspective view of a concentric drilling sleeve.

### DESCRIPTION OF A PREFERRED EMBODIMENT

One form of embodiment of a sleeve system according to the invention will be described below. In the exemplified case, the sleeve system according to the invention comprises, on the one hand, concentric types of sleeve and, on the other, eccentric types of sleeve. In this connection, the terms “concentric” and “eccentric” relate to the location of an axis of symmetry of a through-aperture in the sleeves, relative to the location of an (imaginary) axis of rotation of a sleeve fitted to the handle. The invention could also be used in combination with other types of sleeve.

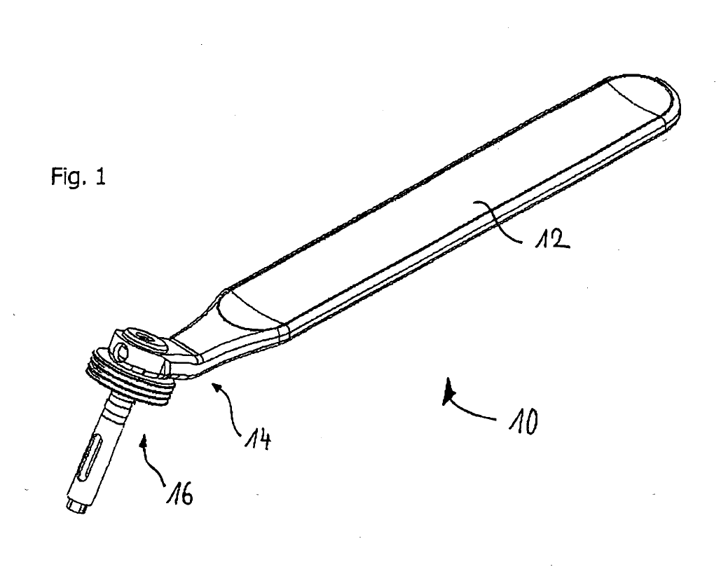

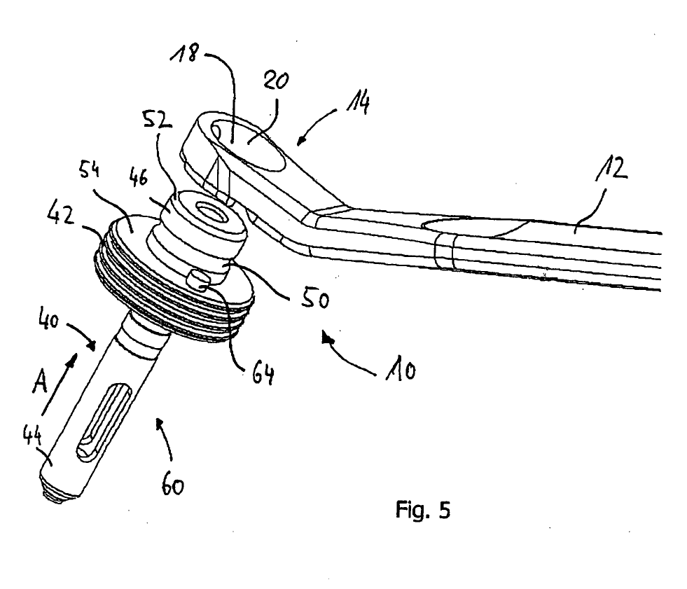

FIG. 1 shows a perspective overall view of a sleeve system 10 according to the invention. Said sleeve system 10 comprises a handle 12 having an angled mounting end 14 for mounting different types of sleeve. The sleeve system 10 also comprises an eccentric sleeve 16 fitted in a rotatable manner to the mounting end 14 of the handle 12. The sleeve system further comprises a concentric sleeve which can be coupled to the mounting end 14 in a rotation-proof manner and which will be explained in greater detail later with reference to FIGS. 5 to 7.

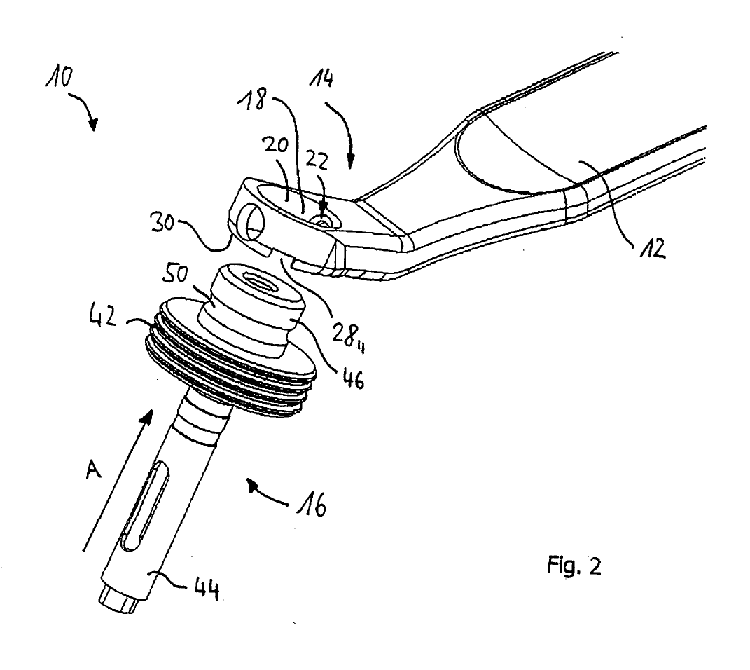

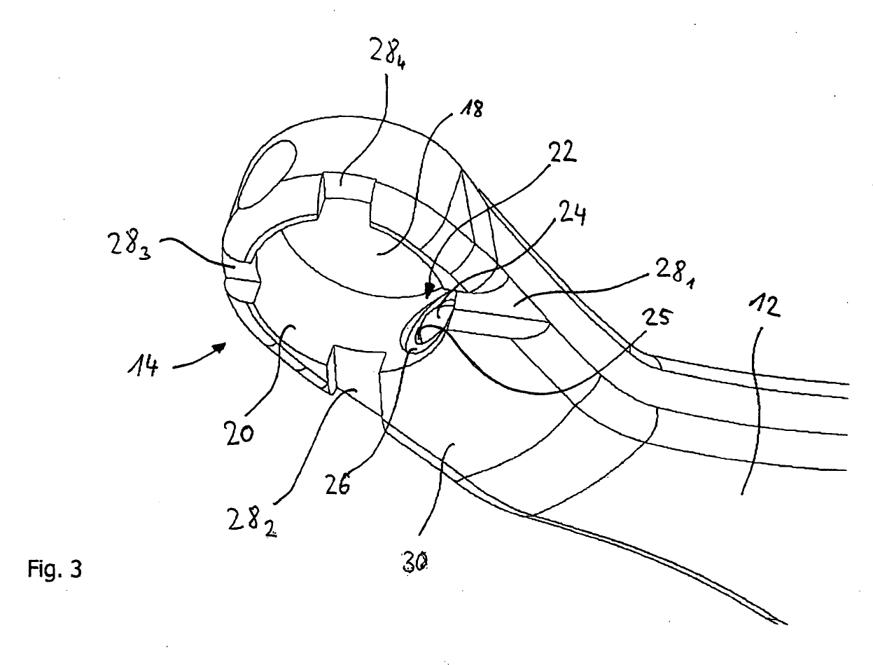

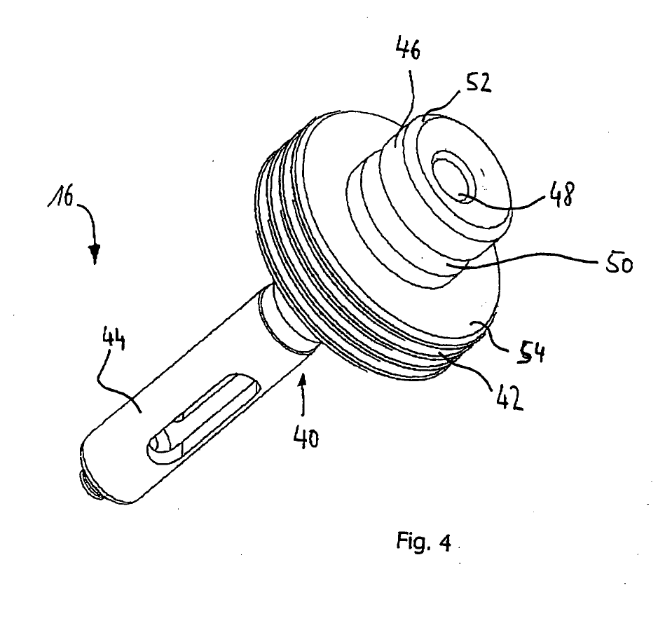

Represented in FIG. 2 is an enlarged perspective view of the mounting end 14 and of the eccentric sleeve 16, prior to the fitting of the latter. FIG. 3 shows a view, which has been enlarged once again, of the mounting end 14, and FIG. 4 shows a view, which has been enlarged once again, of the eccentric sleeve 16.

The make-up and functioning of the mounting end 14 of the handle 12 will be explained in greater detail below with reference to FIGS. 2 and 3. The mounting end 14 possesses a through-aperture 18 into which the sleeves of different type can be introduced. Said through-aperture 18 is bounded by a cylindrical side wall 20. Disposed in the region of said side wall 20 is an arrangement 22 which permits guided rotation of a mounted sleeve of the eccentric type.

The arrangement 22 comprises a guide element in the form of a spherical guide projection 24. Said guide projection 24 protrudes above the side wall 20 in the direction of the through-aperture 18 and is pretensioned in the said direction. For this purpose, the arrangement 22 comprises a spring element which is not represented in the drawings. Like the guide projection 24, said spring element is mounted in a, for example, blind-hole-like bore 25 and rests, on the one hand, against the base of the blind hole 25 and, on the other, against the guide projection 24. In order to prevent the pretensioned guide projection 24 from passing out of the blind hole 25, the latter is closed by a reduction 26 in its internal diameter, the diameter of which reduction is selected so as to be smaller than the external diameter of the spherical guide projection 24.

In the region of the mounting end 14, the handle 12 also possesses an arrangement which interacts with a sleeve of the concentric type (FIGS. 5 to 7) in such a way that rotation of a mounted concentric sleeve relative to the handle 12 is prevented. In the case represented in FIG. 3, the said arrangement comprises a plurality of, in all, four blocking elements 28₁ . . . 28₄ which, in the exemplified case, are groove-shaped. Said blocking elements 28₁ . . . 28₄ are disposed in a recessed manner with respect to an underside 30 of the mounting end 14, which underside is to face towards the sleeves. As can be gathered from FIG. 3, two adjoining blocking elements 28₁ . . . 28₄ each possess an angular interval of 90° with respect to an imaginary axis of rotation, in order to make possible rotation-proof fixing of a concentric sleeve in four defined angular positions with respect to the handle 12.

The make-up and functioning of the eccentric sleeve 16 will now be described with reference to FIGS. 2 and 4. The eccentric sleeve 16 possesses a cylindrical shaft 40 which is divided, by a disc-shaped enlargement in diameter 42, into a lower end 44 that faces away from the mounting end 14 of the handle 12, and an upper end 46 that faces towards said mounting end 14 of the handle 12. The shaft 40 possesses an eccentric through-aperture 48. This means that the axis of symmetry of said through-aperture 48 is offset (towards the left in FIG. 4) with respect to the axis of symmetry of the shaft 40.

The upper end 46 of the shaft is provided, radially on the outside, with a recess extending in the peripheral direction of the shaft 40 in the form of a guide groove 50. Said guide groove 50 interacts with the guide projection 24 on the mounting end 14 as follows: for the purpose of mounting the eccentric sleeve 16 on the handle 12, said sleeve 16 is introduced, in the direction of the arrow A in FIG. 2 and with the upper end 46 of the shaft in front, into the through-aperture 18 in the mounting end 14, the internal diameter of which aperture is slightly larger than the external diameter of the said upper end 46 of the shaft. That end face of the upper end 46 of the shaft which faces towards the mounting end 14 possesses, radially on the outside, a bevelled section 52. When the upper end 46 of the shaft is introduced, the pretensioned guide projection 24 first of all comes into contact with this bevelled region 52 and is thrust by the latter, as the introducing movement continues, counter to the direction of pretensioning, i.e. in the direction of the base of the blind hole 25 already mentioned. As soon as the guide groove 50 in the eccentric sleeve 16 passes, in the course of the introducing movement, into the region of the pretensioned guide projection 24, the latter is moved out of the blind hole 25 in the direction of the guide groove 50 and is able to engage in said guide groove 50 in a latching manner. The eccentric sleeve 16 is thereby coupled to the handle 12 in a captive but rotatable manner.

In the event of the introducing movement continuing, that circular-ring-shaped front face 54 of the enlargement in diameter 42 which faces towards the underside 30 of the mounting end 14 comes into abutment with said underside 30 shortly after the latching of the guide projection 24 into the guide groove 50. This terminates the introducing movement and is a perceptible indication of reliable coupling of the sleeve 16 to the handle 12. If the eccentric sleeve 16 is moved rotationally with respect to the handle 12, for example as a result of clasping the knurled superficies of the enlargement in diameter 42 by means of two fingers and of a subsequent rolling movement of said fingers, the spherical guide projection 24 rolls along in the guide groove 50. This makes possible a largely resistance-free rotation of the sleeve 16 on the one hand, but on the other, the spherical guide projection 24 pretensioned against the base of the guide groove 50 counteracts an unintentional or inadvertent rotation of said sleeve 16.

The interaction of the mounting end 14 of the handle 12 with a concentric sleeve 60 will be described below with reference to FIGS. 3 and 5 to 7.

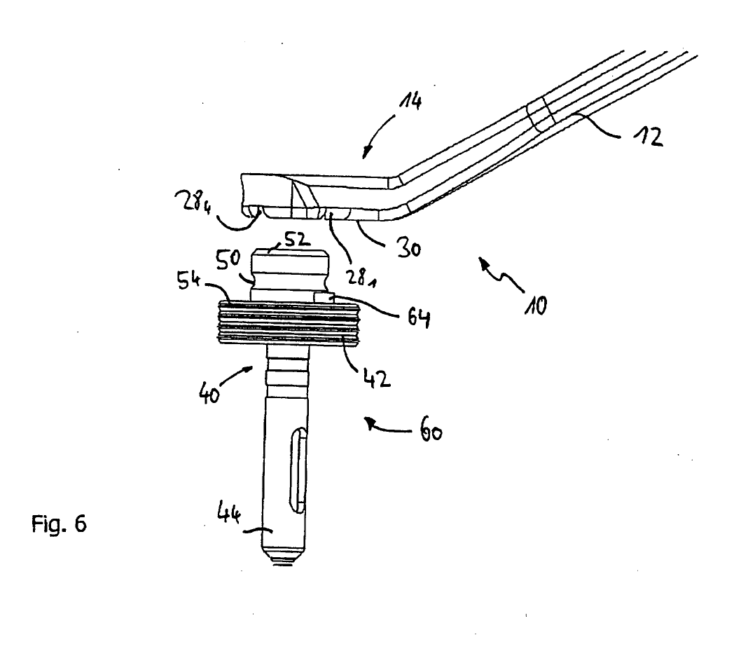

The handle 12, which has already been explained with reference to FIGS. 1 to 3, is represented in FIGS. 5 and 6, together with a concentric sleeve 60. Said concentric sleeve 60 resembles, in many details, the eccentric sleeve 16 that has already been explained. For this reason, elements which correspond to one another have been provided with the same reference numerals.

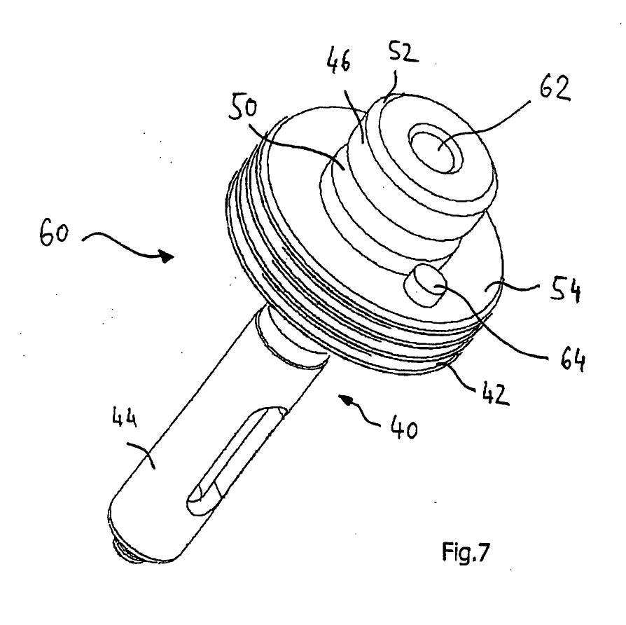

The differences between the eccentric sleeve 16 according to FIGS. 1, 2 and 4 on the one hand, and the concentric sleeve 60 according to FIGS. 5 to 7 on the other, will now be explained with reference to FIG. 7. A first difference consists in the fact that the through-aperture 62 in the concentric sleeve 60 is disposed centrally with respect to the shaft 40. This means that the axis of symmetry of the through-aperture 62 on the one hand, and the axis of symmetry of the shaft 40 on the other, coincide.

Another difference between the eccentric sleeve 16 and the concentric sleeve 60 is the fact that said concentric sleeve 60 is provided, in the region of the end face 54 of the enlargement in diameter 42, with a blocking element in the form of a projection 64 which rises above the surface of said end face 54 and which, in the exemplified case, is cylindrical. This projection 64 interacts with the complementary blocking elements 28₁ . . . 28₄, which have already been explained, on the mounting end 14 of the handle 12, in order to make possible rotation-proof coupling of the concentric sleeve 60 to the handle 12. To that end, the concentric sleeve 60 is introduced by the upper end 46 of its shaft, as already described with regard to the eccentric sleeve 16, into the through-aperture 18 in the mounting end 14 along the arrow A in FIG. 5. However, latching of the pretensioned spherical projection 24 on the mounting end 14 into the guide groove 50 in the concentric sleeve 60, and thereby captive mounting of the latter, is possible only in certain relative angular positions between said concentric sleeve 60 and said mounting end 14. These relative angular positions are predetermined by the location of the groove-shaped blocking elements 28₁ . . . 28₄ in the mounting end 14.

Only if the alignment between the concentric sleeve 60 and the mounting end 14 is selected in such a way that the projection 64 on said concentric sleeve 60 is able to penetrate into one of the grooves 28₁ . . . 28₄ in a form-locking manner, can said concentric sleeve 60 be introduced into the through-aperture 18 in the mounting end 14 to an extent such that latching of the pretensioned guide projection 24 into the guide groove 50, and thereby captive mounting of the concentric sleeve 60, becomes possible. Relative rotation between the concentric sleeve 60 and the handle 12 after the latching of the guide projection 24 into the guide groove 50 is reliably eliminated through the fact that the side faces of the projection 64 on the concentric sleeve 60 abut, or come into abutment in the event of even a minimal rotational movement, against the side faces of one of the groove-shaped recesses 28₁ . . . 28₄.

The sleeves of the sleeve system according to the invention may be used for various purposes in the context of a surgical operation. Thus, said sleeves may serve as a drill-guiding device or as a positioning aid for bone screws or for a screwdriver blade. Other uses which are known, in particular, from the sphere of facial surgery, are likewise possible. The above embodiment of the invention is intended to be an example of the present invention, and alterations and modifications may be effected thereto, by those of ordinary skilled in the art, without departing from the scope of the invention which is defined by the claims handled hereto.

1. A sleeve handle for use in combination with a plurality of different types of sleeve for surgical operations, comprising
an end that is constructed for mounting the different types of sleeve;
a first arrangement for interacting with a mounted sleeve of a first type; and
a second arrangement for interacting with a mounted sleeve of at least one second type;
wherein the first arrangement allows to prevent rotation of the mounted sleeve of the first type, relative to the handle, and the second arrangement allows for a guided rotation of the mounted sleeve of the second type, relative to said handle.

2. The sleeve handle according to claim 1, wherein the first arrangement is constructed so as to interact with a complementary arrangement on the sleeve of the first type in a form-locking or frictional-locking manner.

3. The sleeve handle according to claim 1, wherein the first arrangement comprises at least one blocking element which interacts, for the purpose of eliminating rotation of the mounted sleeve of the first type, with a complementary blocking element on the said sleeve.

4. The sleeve handle according to claim 3, wherein the blocking element on the first arrangement is configured as a recess or as a projection.

5. The sleeve handle according to claim 1, wherein the first arrangement allows for a rotation-proof fixing of the sleeve of the first type in various angular positions with respect to the handle.

6. The sleeve handle according to claim 5, wherein the first arrangement possesses a plurality of blocking elements which are spaced apart in the peripheral direction of an imaginary axis of rotation of the sleeve of the first type.

7. The sleeve handle according to claim 1, wherein the second arrangement possesses a guide element in the form of at least one guide projection or at least one guide groove, which guide element is constructed so as to interact with a complementary guide element on the sleeve of the second type.

8. The sleeve handle according to claim 7, wherein the guide projection is pretensioned in the direction of the guide groove.

9. The sleeve handle according to claim 1, wherein the second arrangement permits captive but rotatable mounting of the sleeve of the second type at the end of the handle.

10. The sleeve handle according to claim 1, wherein the second arrangement permits captive mounting of the sleeve, both of the first and also of the second type, at the end of the handle.

11. The sleeve handle according to claim 1, wherein the handle possesses, at its end which is constructed for mounting the sleeves, an aperture into which said sleeves can be introduced, and wherein the second arrangement is disposed in a region of a side wall of said aperture.

12. A sleeve system for use for surgical operations, comprising
a handle and a plurality of different types of sleeve, wherein one end of the handle is configured for mounting said different types of sleeve, and at least one of the handle and a sleeve of a first type having a first arrangement which prevents rotation of the mounted sleeve of the first type, relative to the handle, and at least one of the handle and a sleeve of a second type having a second arrangement which allows for a guided rotation of the mounted sleeve of the second type, relative to said handle.

13. The sleeve system according to claim 12, wherein the sleeve of the first type possesses an aperture which is concentric with respect to an imaginary axis of rotation of said sleeve.

14. The sleeve system according to claim 12, wherein the sleeve of the second type possesses an aperture which is eccentric with respect to an axis of rotation of said sleeve.

15. The sleeve system according to claim 12, wherein the handle possesses, at its end which is constructed for mounting the sleeves, an aperture into which said sleeves can be introduced, and said sleeves possess an enlargement in diameter which functions as a stop with regard to introduction.

16. A sleeve system for surgical operations, comprising
a handle and a plurality of different types of sleeve, wherein one end of the handle is configured for mounting said different types of sleeve, at least one of the handle and a sleeve of a first type having a first arrangement which prevents rotation of the mounted sleeve of the first type, relative to the handle, and at least one of the handle and a sleeve of a second type having a second arrangement which allows for a guided rotation of the mounted sleeve of the second type, relative to said handle, wherein the sleeve of the first type possesses an aperture which is concentric with respect to an imaginary axis of rotation of said sleeve of the first type and wherein the sleeve of the second type possesses an aperture which is excentric with respect to an axis of rotation of said sleeve of the second type.

17. A sleeve handle for use in surgical operations, the sleeve handle comprising:
a handle portion that allows for a mounting of at least a sleeve of a first type and a sleeve of a second type;
a first arrangement for interacting with a mounted sleeve of a first type such that it allows to prevent a rotation of the sleeve of the first type relative to the handle; and
a second arrangement for interacting with a mounted sleeve of a second type, the second arrangement allowing for a guided rotation of a mounted sleeve of the second type relative to said handle.

18. The sleeve handle according to claim 17, wherein the first arrangement interacts with the sleeve of the first type in a form-locking or frictional-locking manner.

19. The sleeve handle according to claim 17, wherein the second arrangement includes a guide element for rotationally guiding the mounted sleeve of the second type.

20. The sleeve handle according to claim 19, wherein the guide element includes one or more guide projections or one or more guide grooves.
